(19) United States
(12) Patent Application Publication
(10) Pub. No.: US 2006/0224096 A1
(43) Pub. Date: Oct. 5, 2006

(54) Splinter CR/S W

(75) Inventor: Matthew R. Lott, Stow, OH (US)

Correspondence Address: Matthew R. Lott, 1352 Hibbard Drive, Stow, OH, 44224, US

(73) Assignee: Matthew R. Lott, Stow, OH (US)

(21) Appl. No.: 11/093,247

(22) Filed: Mar. 29, 2005

(51) Int. Cl. A61F 5/00

(52) U.S. Cl. 602/21

(57) ABSTRACT

A device for stretching soft tissues and strengthening consisting of a frame that supports a first body segment and applies a dynamic force to a second body segment connected by a joint. The device employs custom-molded thermoplastic cuffs affixed to elastic tubing adjustable for tension and attached to the frame of the device. A custom-molded cuff that includes the digits addresses tightness affecting the muscle tendon units crossing the wrist. The attachment of the rubber tubing at the frame is also adjustable at its terminal end allowing adjustment for the angle of pull in relation to the joint treated.

### REFERENCES CITED [REFERENCED BY]

U.S. Patent Documents

| | | |
|---|---|---|
| 3976057 | August, 1976 | Barclay. |
| 4039183 | August, 1977 | Sakurada. |
| 4229001 | October, 1980 | Roman. |
| 4237873 | December, 1980 | Terry et al. |
| 4310154 | January, 1982 | Kuaffman |
| 4371161 | February, 1983 | Williams. |
| 4441489 | April, 1984 | Evans et al. |
| 4538595 | September, 1985 | Hajianpour. |
| 4538600 | September, 1985 | Hepburn. |
| 4606542 | September, 1986 | Segal. |
| 4612919 | September 1986 | Best. |
| 4665905 | May, 1987 | Brown. |
| 4718665 | January, 1988 | Airy et al. |
| 4869499 | September, 1989 | Schiraldo. |
| 4848326 | July, 1989 | Lonardo. |
| 4930497 | June, 1990 | Saringer |
| 5167612 | December, 1992 | Bonutti. |
| 5213094 | May, 1993 | Bonutti. |
| 5232435 | August, 1993 | Leibinsohn. |
| 5391132 | February, 1995 | Greenwald. |
| 5653680 | August, 1997 | Cruz. |

### OTHER REFERENCES

2005 Sammons Preston Rolyan Professional Rehab Catalogue (selected pages) Sammons Preston Rolyan, 4 Sammons Court, Bolingbrook, L 60440-499

### BACKGROUND OF THE INVENTION

1. Technical Field

The present invention includes a combination of custom-molded cuffs and a device for delivering an adjustable dynamic force across a contracted joint to improve range of motion, soft tissue extensibility, and strength in the human body.

2. Description of the Prior Art

When range of motion of a joint is lost secondary to surgical intervention, trauma, disease, or immobilization, soft tissues including skin, tendons, muscles, ligaments, nerves, blood vessels and connective tissues contract and limit the function of the joint. Concomitant weakness of associated muscle tendon units further limits functional use of the joint. While various devices are available to address deficits in joint range of motion none of the prior art discloses a device according to the present invention. For example, Barclay U.S. Pat. No. 3,976,057 discloses an adjustable exercise device comprised of an upper and lower strap surrounding the leg above and below a knee joint and utilizing compressed air for resistance. Sakurada U.S. Pat. No. 4,039,183 shows a device for strengthening the human wrist via proximal and distal cuffs attached by a spring tension unit. Terry, et al. U.S. Pat. No. 4,237,873 discloses a frame device using cuffs attached to an arm to control tremors associated with cerebral palsy and using resistance via a piston and rod assembly to provide resistance to the body segment. Williams U.S. Pat. No. 4,371,161 describes a device for improving ankle dorsiflexion using a proximal segment cuff attached to elastic tubing having its free end attached to a cuff around the foot. Evans U.S. Pat. No. 4,441,489 discloses a splint design consisting of a proximal portion and distal portion connected with a spring to improve motion of a stiff finger joint. Hajianpour U.S. Pat. No. 4,538,595 discloses a passive exercise device for improving joint motion using a drive motor and gear assembly. Segal U.S. Pat. No. 4,606,542 discloses a limb exercise device consisting of a hinged brace using elastic members to provide resistance to joint movement. Best U.S. Pat. No. 4,612,919 describes a brace fitting above and below a joint and providing static adjustable force to gradually improve joint motion. Brown U.S. Pat. No. 4,665,905 discloses a wire frame brace fitting over a limb connected by a spring to improve joint motion. Lonardo U.S. Pat. No. 4,848,326 discloses a device for correcting a knee joint contracture using a pair of rod assemblies to provide static progressive stretch to the joint. Schiraldo U.S. Pat. No. 4,869,499 discloses a toe exercise device consisting of a proximal segment brace surrounding the limb attached to an elastic band and connecting to a cuff on a toe to improve toe dorsiflexion and strengthen the toe flexor muscles. Saringer U.S. Pat. No. 4,930,497 discloses an apparatus to provide continuous passive motion to a lower limb using a brace connected by a hinge and resting on a base and using an electric motor to mobilize the limb in the brace. Leibinsohn U.S. Pat. No. 5,232,435 discloses a wire frame brace connected by a pivot and using spring tension to stretch an elbow joint into extension. Bonutti U.S. Pat. No. 5,167,612 discloses a device for mobilizing a joint in flexion or dorsiflexion while providing a distractive force across the joint using a brace secured above and below the joint. Liu U.S. Pat. No. 5,364,323 discloses a wrist exercise device consisting of a proximal and distal brace connected with a spring tension device to provide resistance to the forearm muscles. Greenwald U.S. Pat. No. 5,391,132 discloses an exercise device for the shoulder using elastic tubing for resistance. Cruz U.S. Pat. No. 5,653,680 discloses a dynamic orthotic device to improve wrist motion via spring tension. Splints sold under the Trademark DYNASPLINT by Dynasplint Systems, Inc. provide an adjustable, low profile dynamic splint to improve wrist dorsiflexion and flexion. Splints sold under the Trademark MERIT through the 2005 Sammons Preston Rolyan Professional Rehab Catalogue provide static progressive splints using a static line attached to a knob that is turned to increase tension and improve wrist flexion and dorsiflexion. Each of the above-identified prior art devices and each of the devices in use at the present time do not provide a combination of attributes found in the present invention. These attributes include custom-molded cuffs that provide a conforming fit that minimize pressure areas by dispersing forces evenly across soft tissues improving both comfort and wearing time and preventing injury to skin. Another attribute of the present invention is the ability to improve joint range of motion in two planes with a single unit. Further attributes include the ability to adjust and maintain a 90-degree angle of pull to the joint, dynamic tension, and the ability to perform stretching of the digital flexors. Another attribute of the present device is the high profile design allowing a greater range of tension adjustment. Attributes also include the ability to apply strengthening, contract and relax, and dynamic progressive force to the specified muscles and joint. Other attributes of the present invention include a lightweight design, tension adjustment without tools, and tension adjustment using the free hand. In comparison to the prior art, the present device uses inexpensive materials and lacks complex, machined parts, which maintains a low cost and facilitates part replacement.

### SUMARY OF THE INVENTION

The present invention comprises a device for increasing the range of motion of a stiff wrist joint, strengthening muscles in the vicinity of the wrist, and improving flexibility of muscles and tendons associated with wrist and digital motion. The user's forearm rests in the device with the palm up (supinated) or palm down (pronated) to improve wrist dorsiflexion or flexion respectively. A cooperating hook and loop strap commonly known by the trademark “VELCRO” is placed across the forearm and secured to the opposite side of the forearm rest. A VELCRO loop elbow strap wrapped around the user's arm above the elbow is secured to a post that has an adhesive VELCRO hook affixed. A low temperature thermoplastic cuff is molded to the user's hand. A VELCRO loop strap affixed to the elastic tubing is secured at one side of the cuff. The opposite end of the elastic tubing is tied to another VELCRO loop strap and the elastic tubing is passed through a carabineer on the base of the device's frame and back to the cuff where the tension can be adjusted via the VELCRO hook and loop strap. Sliding the carabineer forward or backward along the base of the device varies the angle of pull to the wrist joint based on the joint's range of motion. The user allows the elastic band to stretch the wrist for a comfortable period of time followed by movements against the resistance of the device to strengthen muscles. This method of contracting the muscles on one side of the joint assists in the relaxation of muscles on the opposing side of the joint to improve flexibility via reciprocal inhibition. The same cuff is used for improving both wrist dorsiflexion and wrist flexion. A custom molded pan cuff is available for addressing extrinsic flexor tightness. The pan cuff is placed on the palm of the supinated hand and maintains the fingers in an extended position. The elastic band is secured to the pan cuff via cooperating hook and loop straps and adjusted for tension. The user allows the elastic band to pull the wrist into dorsiflexion stretching the wrist joint in addition to muscles crossing the wrist including flexor digitorum profundus and flexor digitorum communis.

The high profile design of the present invention allows greater adjustability of resistance and facilitates a 90 degree angle of pull in relation to the joint treated thereby reducing shear stresses across the joint and improving comfort and wearing tolerance.

For appropriate clients, the device is issued for home use minimizing the so-called “rebound effect” where improvements in motion are lost between therapy appointments.

The present invention improves the efficiency of the clinical staff allowing the therapist to attend to other tasks while the patient is treated in the device.

The present invention is versatile and can provide various methods of treating contracted joints including low-load prolonged stretch, contract and relax, strengthening, and progressive dynamic loading.

The present invention utilizes low temperature thermoplastic material molded to the frame of the device and to each client's hand to ensure even distribution of pressure, improve wearing comfort and tolerance, and minimize the possibility of injury.

The device does not require the user to actively hold or maintain a grasp on the cuff(s) and is therefore suitable for improving range of motion and importantly strength of the wrist when finger function is limited or absent. This is desirable since the ability to flex the digits is dependent on the ability to extend and maintain extension of the wrist.

### BRIEF DESCRIPTION OF THE DRAWINGS

The following features of the present invention will become apparent to one familiar with the art based on the description of the invention with reference to the subsequent drawings, wherein:

FIG. 1 is a view of a device in accordance with the present invention without “VELCRO” straps, custom-molded cuff, adjustable tension device, or user's forearm.

FIG. 2 is a view of a pronated human forearm (represented by dotted lines) with VELCRO elbow strap and VELCRO wrist strap in place with the custom-molded cuff assembly designed to improve wrist flexion with adjustable VELCRO tension strap.

FIG. 3 is a view of a supinated human forearm in the device with VELCRO elbow strap and VELCRO wrist strap in place and the custom-molded cuff assembly designed to improve wrist dorsiflexion with adjustable VELCRO tension strap.

FIG. 4 is a view of a supinated human forearm in the device with VELCRO elbow strap and VELCRO wrist strap in place with the custom-molded pan splint assembly designed for decreasing extrinsic flexor tightness with adjustable VELCRO tension strap.

### DESCRIPTION OF PREFERRED EMBODIMENTS OF THE INVENTION

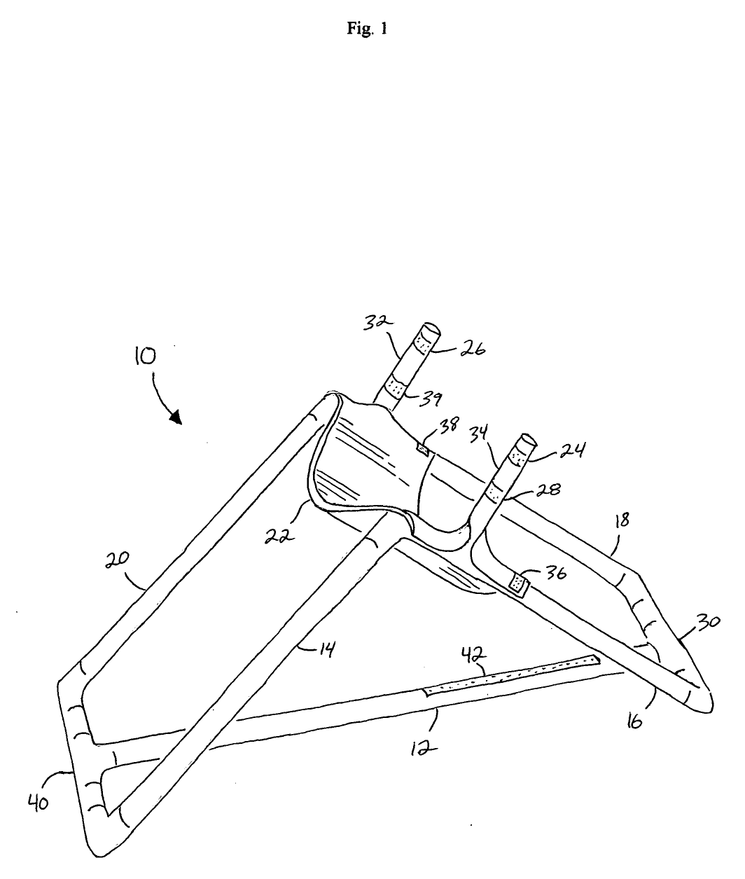

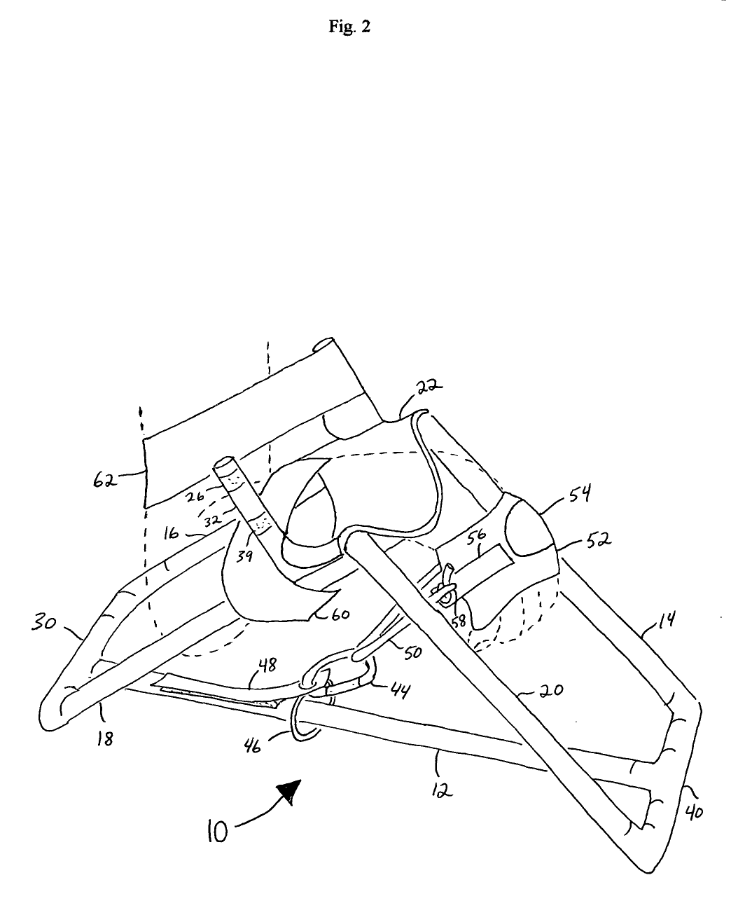

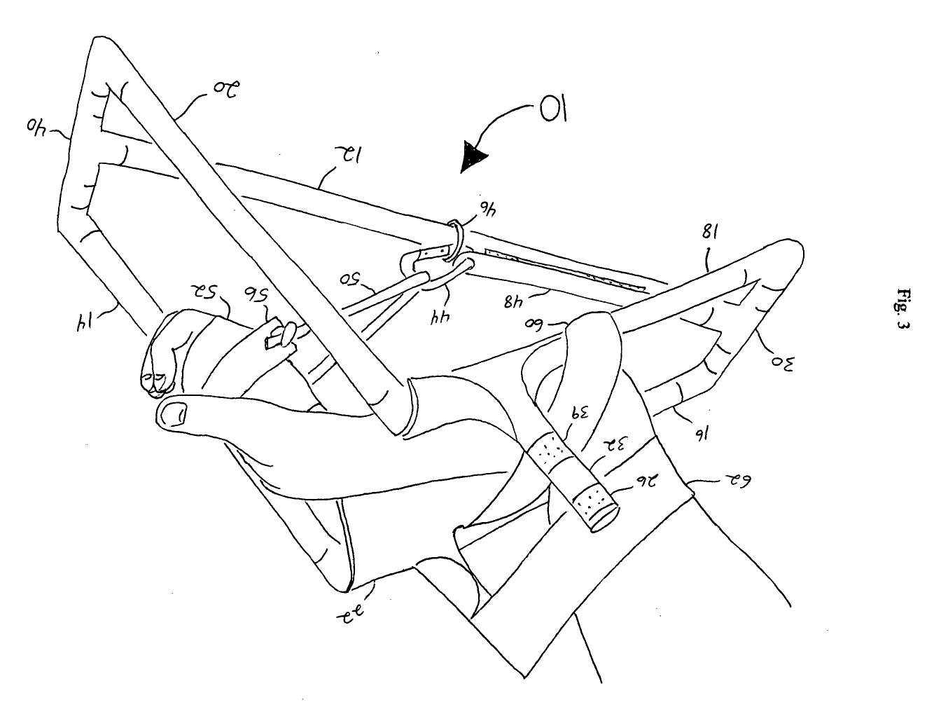

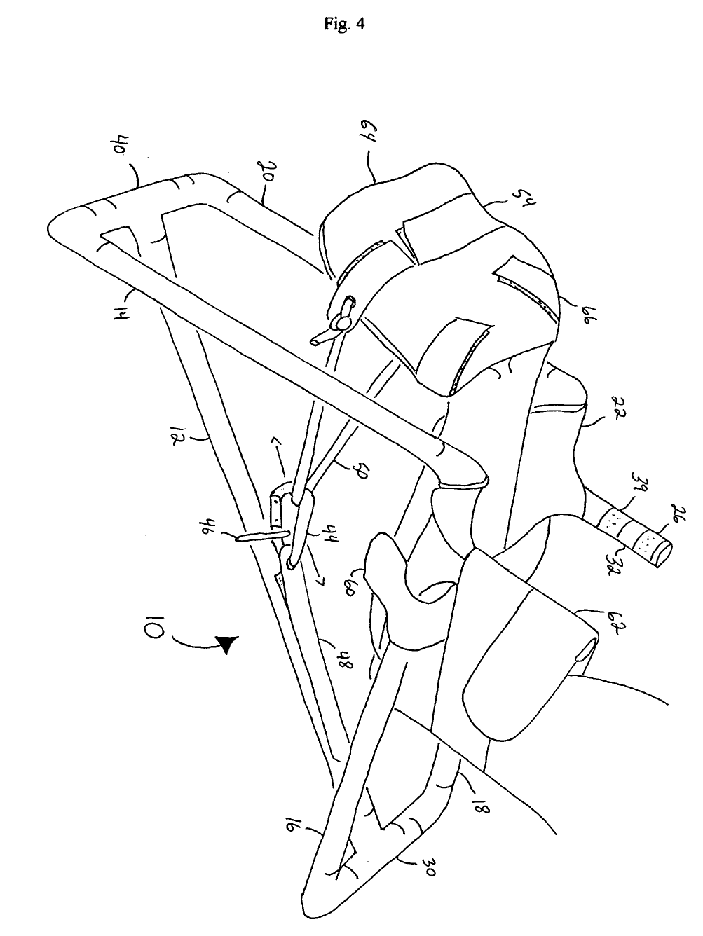

Referring to the drawings wherein like parts are designated with the same reference numeral throughout the four views, FIG. 1 demonstrates a frame 10 consisting of a strut 12 spanning the base of the device and serving as an attachment point for the clip 44 and elastic tubing 50 via a VELCRO strap that is adjustable along the length of the strut 12 as depicted in FIG. 4. Referring now to FIG. 1, the strut 12 connects at one end to strut 40 and at its opposite end to strut 30 to form the base of the frame. The strut 40 is connected at either end to forward support struts 14 and 20. The strut 30 is connected at either end to rear support struts 16 and 18. The forward strut 16 is connected to the rear support strut 14 and the forward strut 20 is connected to the rear support strut 18. The forward and rear support struts form an “A” shaped frame spanned by a sufficiently rigid forearm rest 22. Connecting to the rear support struts 16 and 18 the posts 34 and 32 serve as attachment points for the VELCRO elbow strap 62 as illustrated in FIGS. 2, 3, and 4. The VELCRO elbow support strap 62 attaches to the posts 16 and 18 using adhesive VELCRO hook 39 and 26. Affixed to the forearm rest 22 are adhesive VELCRO hook 36 and 38 that cooperate with the VELCRO forearm strap 60 as illustrated in FIGS. 2, 3, and 4. FIG. 2 illustrates a forearm (shown in dotted lines) resting in the device with the palm rotated downward in a pronated position with the custom-molded cuff 52 and the elastic tubing 50 attached to the cuff 52 on the radial side by a VELCRO loop strap 54 and on the ulnar aspect of the cuff by VELCRO loop strap 56. The elastic tubing 50 is shown tied to the strap 56 by a simple square knot 58. The VELCRO loop straps 56 and 54 allow the user to adjust the amount of force applied through the cuff to the user's wrist. The elastic tubing 50 passes through the clip 44. The clip 44 is attached to the frame 10 at the strut 12 by a metal ring 46 allowing the angle of force to be adjusted by the VELCRO loop strap 48 that is attached at one end to the clip 44 and at the opposite end on the strut 12 via the adhesive VELCRO hook 42. The VELCRO elbow strap 62 is attached at strut 32 forming a supportive sling around the user's arm. The VELCRO forearm strap 60 stabilizes the forearm in the semicircular forearm rest 22. FIG. 3 illustrates a supinated forearm resting in the device 10 with the custom-molded cuff 52 attached to the elastic tubing 50 demonstrating the device's capability to improve wrist dorsiflexion. FIG. 4 illustrates a supinated forearm resting in the device 10 with palm facing up with custom-molded pan splint 64 in place with the digits in extension while wrist is dorsiflexed thereby stretching the extrinsic flexor muscles. The VELCRO loop strap 66 encircles the wrist preventing distal migration of the pan splint 64. Other fastening means including snaps, rivets, and buckles may be used to secure the forearm in the device and provide a means of adjusting tension on the cuffs. Although the cuffs and forearm rest are custom-molded in the present device, it is conceivable that pre-formed cuffs or forearm rests of varying sizes could be used. The custom-molded cuff 52 is used for both improving dorsiflexion and flexion of the wrist and it is conceivable that adjustments in the shape and size of the cuffs could be made without departing from the spirit of the present invention. It is also conceivable that the fingers, when taped in a flexed position as when making a fist, while in the cuff 52, could be used to improve flexibility of the digital extensors. It should also be noted that the elastic tubing on any of the cuffs could be replaced with an adjustable static line and the present invention is intended to cover this.

Note that the present invention can be used to extend or flex other joints of the body such as the ankle, knee, or elbow with adjustment of the present orthosis 10 varied to address the particular application. For example, the size of the frame could easily be increased and the thermoplastic cuff material molded to fit the elbow, knee, and ankle joints.

It should be understood that the arrangement of the frame of the invention, body segment supports, straps, cuffs, and elastic bands are not the only possible arrangement of those elements. The invention imagines that other arrangements of these similarly functional elements are quite suitable, and thus, the invention is intended to cover any such similar arrangement including the addition of a motor connected to the cuff and frame for continuous passive motion.

It should be noted that the materials composing the invention are not the only suitable materials for the construction of the device. The invention contemplates that other materials are suitable for the construction, and thus, the invention is intended to cover any such materials.

Although the present invention has been described with preferred embodiments, those skilled in the art will perceive improvements, enhancements, variations and modifications. Such improvements, enhancements, variations and modifications are considered to be within the scope and purview of the present invention.

What I claim is:

1. A device for increasing range of relative motion between first and second body segments interconnected by a joint, said method comprising the steps of securing the first body segment in a frame consisting of a semicircular support curved along its entire length and molded to conform to the body segment and a custom-molded cuff secured to the second body segment that is connected to the frame via an adjustable elastic line to stretch soft tissues, improve range of motion, and strengthen muscles in the vicinity of the joint.

2. A method set forth in claim 1 wherein said steps of stretching soft tissue connected with a joint are applied to improve joint flexion.

3. A method set forth in claim 1 wherein said steps of stretching soft tissue connected with a joint are applied to improve joint extension.

4. A method set forth in claim 1 wherein said steps of stretching soft tissue connected with a joint are applied to improve flexibility of muscle tendon units that cross the wrist joints and flex the digits.

5. A method set forth in claim 1 wherein the vector of force application between the custom-molded cuff and device frame is adjustable.
